(19) United States
(12) Patent Application Publication
(10) Pub. No.: US 2026/0328762 A1
(43) Pub. Date: Oct. 22, 2026

(54) FELINE TRIPLE ANTIBODY CHROMATOGRAPHIC TEST STRIP, PREPARATION METHOD THEREFOR, USE THEREOF

(71) Applicant: Beijing Jinnuo Biotech Co.,Ltd., Beijing (CN)

(72) Inventors: Jingliang WEN, Beijing (CN); Pujun LI, Beijing (CN); Dandan HUANG, Beijing (CN); Xingliang WU, Beijing (CN); Xun YANG, Beijing (CN); Xinrui ZHANG, Beijing (CN); Yating GUO, Beijing (CN); Wenpeng ZHU, Beijing (CN); Yinbiao MENG, Beijing (CN); Jingru CHEN, Beijing (CN); Sudan WANG, Beijing (CN); Yuqi JIA, Beijing (CN); Lei FANG, Beijing (CN); Peng FU, Beijing (CN); Yaling WANG, Beijing (CN); Xin ZHANG, Beijing (CN)

(73) Assignee: Beijing Jinnuo Biotech Co.,Ltd., Beijing (CN)

(21) Appl. No.: 19/649,682

(22) Filed: Apr. 16, 2026

(30) Foreign Application Priority Data
Apr. 18, 2025 (CN) 202510490471.3

(51) Int. Cl. G01N 33/543; G01N 33/53; G01N 33/532; G01N 33/552; G01N 33/569; G01N 33/68

(52) U.S. Cl. CPC G01N 33/54387; G01N 33/5302; G01N 33/532; G01N 33/54306; G01N 33/552; G01N 33/56983; G01N 33/68; G01N 2333/015

(57) ABSTRACT

A feline triple antibody chromatographic test strip is used for biological detection. The feline triple antibody chromatographic test strip comprises a PVC backing plate; the PVC backing plate is provided with a sample pad, a conjugate pad, a nitrocellulose membrane, and an absorbent pad; the nitrocellulose membrane is provided with a test T line and a control C line; and the conjugate pad contains a feline panleukopenia virus VP2 protein probe labeled with SiO₂-coated quantum dot microspheres emitting a light at a wavelength of 565 nm, a feline calicivirus VP1 protein probe labeled with SiO₂-coated quantum dot microspheres emitting a light at a wavelength of 525 nm, and a feline herpesvirus gD protein probe labeled with SiO₂-coated quantum dot microspheres emitting a light at a wavelength of 610 nm. The test strip exhibits better stability and yields more accurate detection results.

### CROSS-REFERENCE TO RELATED APPLICATION

This patent application claims the benefit and priority of Chinese Patent Application No. 202510490471.3 filed with the China National Intellectual Property Administration on Apr. 18, 2025, the disclosure of which is incorporated by reference herein in its entirety as part of the present application.

### TECHNICAL FIELD

The present application relates to the field of biological detection technology, in particular to a feline triple antibody chromatographic test strip, a preparation method therefor, and the use thereof.

### BACKGROUND

Feline panleukopenia is a highly contagious disease caused by infection with feline panleukopenia virus (also known as feline parvovirus); feline calicivirus and feline herpesvirus are common pathogens causing feline respiratory diseases. The incidence and mortality rates of the above three diseases are relatively high, posing a serious threat to feline health. At present, the prevention and control of the above three diseases mainly adopt vaccine immunization, and the key to judging the success of vaccine immunization lies in detecting whether corresponding viral antibodies are produced in felines. As an important tool for evaluating the immune status of felines, detection of feline triple antibody is of great significance for the prevention and treatment of the diseases.

At present, the common method for detecting feline triple antibody in clinical diagnosis is the Dot-ELISA method. Although this method employs a simplified ELISA method, the detection operation process is relatively cumbersome and time-consuming, requiring the detection operator to perform operations at multiple time points. Moreover, different personnel have a relatively large deviation in color discrimination, and judging the results only by the naked eye may lead to significant errors, resulting in insufficient accuracy of the detection results. In addition, when using the above method to detect clinical samples, there is also a problem of a relatively high false positive rate.

Therefore, there is a need to provide a chromatographic test strip capable of simultaneously detecting feline panleukopenia virus antibody, feline calicivirus antibody, and feline herpesvirus antibody, so as to improve the sensitivity, detection efficiency, and convenience of the detection of feline virus antibodies, and reduce false positives in detection results.

### SUMMARY

In order to improve the sensitivity, detection efficiency, and convenience of the detection of feline virus antibodies, and to reduce false positives in detection results, the present application provides a feline triple antibody chromatographic test strip, a preparation method therefor, and the use thereof.

In a first aspect, the present application provides a feline triple antibody chromatographic test strip, which uses the following technical solutions:
A feline triple antibody chromatographic test strip includes a PVC backing plate; the PVC backing plate is provided with a sample pad, a conjugate pad, a nitrocellulose membrane, and an absorbent pad; the nitrocellulose membrane is provided with a test T line and a control C line; and the sample pad is provided with a sample well;
   the conjugate pad contains a feline panleukopenia virus VP2 protein probe labeled with SiO₂-coated quantum dot microspheres emitting at a wavelength of 565 nm, a feline calicivirus VP1 protein probe labeled with SiO₂-coated quantum dot microspheres emitting at a wavelength of 525 nm, and a feline herpesvirus gD protein probe labeled with SiO₂-coated quantum dot microspheres emitting at a wavelength of 610 nm;
   the test T line is coated with a goat anti-feline IgG polyclonal antibody, and the control C line is coated with a mouse anti-VP2 protein monoclonal antibody, a mouse anti-VP1 protein monoclonal antibody, and a mouse anti-gD protein monoclonal antibody.

The present application provides a feline triple antibody chromatographic test strip, which judges whether the test sample is positive or negative by detecting fluorescence values emitted by SiO₂-coated quantum dot microspheres. The feline triple antibody chromatographic test strip has the advantages of high sensitivity, low false positive rate, short time consumption, simple operation, detection results not being subject to the subjective influence of the tester, etc., is suitable for places such as pet hospitals, animal quarantine departments, and farms, and can quickly and conveniently detect whether felines contain feline panleukopenia virus antibody, feline calicivirus antibody, and feline herpesvirus antibody, thereby determining whether the felines have been successfully immunized by vaccination, and providing strong support for the prevention of feline diseases.

The present application adopts SiO₂-coated quantum dot microspheres. The SiO₂ on the surfaces of the quantum dot microspheres can provide a chemical and physical barrier, effectively reducing the fluorescence attenuation of the quantum dot microspheres, greatly improving the fluorescence stability of the quantum dot microsphere test strip, and making the detection results of the prepared test strip more accurate.

During the use of the feline triple antibody chromatographic test strip provided by the present application, when a test sample contains feline panleukopenia virus antibody, feline calicivirus antibody, and feline herpesvirus antibody, the above antibodies form complexes with the feline panleukopenia virus VP2 protein probe labeled with SiO₂-coated quantum dot microspheres, the feline calicivirus VP1 protein probe labeled with SiO₂-coated quantum dot microspheres, or the feline herpesvirus gD protein probe labeled with SiO₂-coated quantum dot microspheres emitting at different wavelengths in the conjugate pad. The complexes move sequentially along the nitrocellulose membrane to the test T line and the control C line, and bind to the goat anti-feline IgG polyclonal antibody on the test T line, causing the test T line to develop color at a given wavelength, thereby judging whether the sample is positive or negative; when the unbound protein probes labeled with quantum dot microspheres move to the control C line, they can react with the mouse anti-VP2 protein monoclonal antibody, the mouse anti-VP1 protein monoclonal antibody, and the mouse anti-gD protein monoclonal antibody on the control C line to form a reaction line, thereby proving that the result of the test T line is true and reliable.

In the present application, the SiO₂-coated quantum dot microspheres emitting a light at a wavelength of 525 nm are green, the SiO₂-coated quantum dot microspheres emitting a light at a wavelength of 565 nm are yellow, and the SiO₂-coated quantum dot microspheres emitting a light at a wavelength of 610 nm are red. The above three types of quantum dots have narrow emission peaks and do not interfere with each other.

In a second aspect, the present application provides a method for preparing a feline triple antibody chromatographic test strip.

The method for preparing a feline triple antibody chromatographic test strip includes the following steps: preparing a conjugate pad, preparing a nitrocellulose membrane, preparing a sample pad, and assembling the feline triple antibody chromatographic test strip;
where a method for preparing the conjugate pad includes: activating SiO₂-coated quantum dot microspheres emitting lights at wavelengths of 565 nm, 525 nm, and 610 nm, then respectively adding feline panleukopenia virus VP2 protein, feline calicivirus VP1 protein, and feline herpesvirus gD protein for antigen labeling, and performing blocking and resuspension to respectively obtain a solution of VP2 protein labeled with SiO₂-coated quantum dot microspheres, a solution of VP1 protein labeled with SiO₂-coated quantum dot microspheres, and a solution of gD protein labeled with SiO₂-coated quantum dot microspheres; diluting the solution of VP2 protein labeled with SiO₂-coated quantum dot microspheres, the solution of VP1 protein labeled with SiO₂-coated quantum dot microspheres, and the solution of gD protein labeled with SiO₂-coated quantum dot microspheres to a final concentration of 10 v/v % to 20 v/v %, and then sequentially spraying the diluted solutions onto a glass fiber surface at a spraying amount of 8±1 μL/cm to obtain the conjugate pad containing VP2 protein labeled with SiO₂-coated quantum dot microspheres, VP1 protein labeled with SiO₂-coated quantum dot microspheres, and gD protein labeled with SiO₂-coated quantum dot microspheres.

In the present application, the spraying sequence of the solution of VP1 protein labeled with SiO₂-coated quantum dot microspheres, the solution of VP2 protein labeled with SiO₂-coated quantum dot microspheres, and the solution of gD protein labeled with SiO₂-coated quantum dot microspheres is sequential spraying, that is, after one protein solution is sprayed, vacuum lyophilization is performed, and then another protein solution is sprayed; where the temperature of the vacuum lyophilization is −20° C. to −30° C., and the time of the vacuum lyophilization is 40-54 h.

Optionally, a method for preparing the nitrocellulose membrane includes: first, drawing a test T line and a control C line on the surface of a nitrocellulose membrane; and then, using a dispenser, sequentially spraying the diluted solution of mouse anti-VP2 protein monoclonal antibody, the diluted solution of mouse anti-VP1 protein monoclonal antibody, and the diluted solution of mouse anti-gD protein monoclonal antibody onto the control C line, and spraying the diluted solution of goat anti-feline IgG polyclonal antibody onto the test T line, followed by vacuum drying to obtain the nitrocellulose membrane;

where the concentrations of the diluted solution of mouse anti-VP2 protein monoclonal antibody, the diluted solution of mouse anti-VP1 protein monoclonal antibody, and the diluted solution of mouse anti-gD protein monoclonal antibody are each 0.95-1.05 μg/μL; the concentration of the diluted solution of goat anti-feline IgG polyclonal antibody is 0.75-0.85 μg/μL; the spraying amounts are each 0.8-1.2 μL/cm.

Optionally, in the nitrocellulose membrane, a distance between the test T line and the control C line is 4±0.5 mm. Optionally, in the spraying operation of the control C line, after one diluted solution is sprayed, vacuum drying is performed, and then another diluted solution is sprayed.

Optionally, in the spraying operation of the control C line, the temperature of the vacuum drying is 37° C.±5° C., and the time of the vacuum drying is 24-72 h.

Optionally, a method for preparing the sample pad includes: first, preparing a sample pad treatment solution, then soaking a glass fiber in the sample pad treatment solution for 20-25 min, and drying the glass fiber to obtain the sample pad;
a method for preparing the sample pad treatment solution includes: dissolving bovine serum albumin and gelatin in a PBS solution, then adding Tween-20, and filtering the resulting solution to obtain the sample pad treatment solution.

In a third aspect, the present application provides a feline triple antibody chromatographic test strip kit.

The feline triple antibody chromatographic test strip kit includes the feline triple antibody chromatographic test strip or a feline triple antibody chromatographic test strip obtained by the method for preparing a feline triple antibody chromatographic test strip; and a sample diluent;
where the sample diluent includes the following components in mass content: 0.8%-1.2% of PEG2000, 0.8%-1.2% of Tween-20, 0.02%-0.04% of Proclin300, 0.1%-0.5% of thiourea, 0.05%-0.2% of deferoxamine, and the balance being a 100 mM Tris-HCl buffer at pH 8.0.

In the present application, by using the components including the above mass contents as the sample diluent, especially the use of thiourea and deferoxamine, it is possible to neutralize oxidizing substances in a sample, reduce the interference of iron ions in the sample, and inhibit the activity of pseudoperoxidase in hemoglobin, thereby greatly reducing the false positive rate in feline clinical whole blood samples and improving the accuracy of the detection results.

In some embodiments, the content of thiourea may be 0.1%-0.2%, 0.1%-0.3%, 0.1%-0.4%, 0.2%-0.3%, 0.2%-0.4%, 0.2%-0.5%, 0.3%-0.4%, 0.3%-0.5%, or 0.4%-0.5%.

In a specific embodiment, the content of thiourea may also be 0.1%, 0.2%, 0.3%, 0.4%, or 0.5%.

In some embodiments, the content of deferoxamine may be 0.05%-0.1%, 0.05%-0.15%, 0.1%-0.15%, 0.1%-0.2%, or 0.15%-0.2%.

In a specific embodiment, the content of deferoxamine may also be 0.05%, 0.1%, 0.15%, or 0.2%.

In a fourth aspect, the present application provides the use of the feline triple antibody chromatographic test strip, a feline triple antibody chromatographic test strip obtained by the method for preparing a feline triple antibody chromatographic test strip, or the feline triple antibody chromatographic test strip kit in the detection of feline panleukopenia virus antibody, feline calicivirus antibody, and feline herpesvirus antibody, where a method of using the feline triple antibody chromatographic test strip/kit includes the following steps: diluting a test sample 80-120 fold with the sample diluent, adding the diluted test sample into the sample well of the feline triple antibody chromatographic test strip, and leaving the test strip to stand for 10-15 min; and then reading T/(T+C) values at emission wavelengths of 565 nm, 525 nm, and 610 nm respectively using a fluorescence analyzer, and judging whether the test sample is positive or negative.

In the present application, whether a test sample is positive or negative is judged by detecting fluorescence values at different emission wavelengths, which has the advantages of high sensitivity, no influence from subjective factors, etc., and higher accuracy of the detection results. Specifically, at an emission wavelength of 525 nm, when the T/(T+C) value is >0.1, it indicates that the test sample contains feline calicivirus antibody; at an emission wavelength of 565 nm, when the T/(T+C) value is >0.1, it indicates that the test sample contains feline panleukopenia virus antibody; and at an emission wavelength of 610 nm, when the T/(T+C) value is >0.1, it indicates that the test sample contains feline herpesvirus antibody.

Optionally, the test sample is selected from the group consisting of serum, whole blood, and saliva.

In summary, the present application has the following beneficial effects:
1. The present application provides a feline triple antibody chromatographic test strip, which judges whether a test sample is positive or negative by detecting fluorescence values emitted by SiO₂-coated quantum dot microspheres. The feline triple antibody chromatographic test strip has the advantages of high sensitivity, low false positive rate, short time consumption, simple operation, detection results not being subject to the subjective influence of the tester, etc., is suitable for places such as pet hospitals, animal quarantine departments, and farms, and can quickly and conveniently detect whether feline panleukopenia virus antibody, feline calicivirus antibody, and feline herpesvirus antibody are produced in felines, thereby judging whether the felines have been successfully immunized by vaccination, and providing strong support for the prevention of feline diseases.
2. In the present application, by adjusting the labeling relationship between the three wavelengths of SiO₂-coated quantum dot microspheres and the three proteins to: a feline panleukopenia virus VP2 protein probe labeled with SiO₂-coated quantum dot microspheres emitting a light at a wavelength of 565 nm, a feline calicivirus VP1 protein probe labeled with SiO₂-coated quantum dot microspheres emitting a light at a wavelength of 525 nm wavelength, and a feline herpesvirus gD protein probe labeled with SiO₂-coated quantum dot microspheres emitting a light at a wavelength of 610 nm, the prepared feline triple antibody chromatographic test strip can achieve simultaneous detection of feline panleukopenia virus antibody, feline calicivirus antibody, and feline herpesvirus antibody, and has high accuracy and good stability in the detection results.

### DETAILED DESCRIPTION OF THE EMBODIMENTS

The present application provides a feline triple antibody chromatographic test strip including a PVC backing plate; the PVC backing plate is provided with a sample pad, a conjugate pad, a nitrocellulose membrane, and an absorbent pad; and the nitrocellulose membrane is provided with a test T line and a control C line;
the conjugate pad contains a feline panleukopenia virus VP2 protein probe labeled with SiO₂-coated quantum dot microspheres emitting a light at a wavelength of 565 nm, a feline calicivirus VP1 protein probe labeled with SiO₂-coated quantum dot microspheres emitting a light at a wavelength of 525 nm, and a feline herpesvirus gD protein probe labeled with SiO₂-coated quantum dot microspheres emitting a light at a wavelength of 610 nm;
the test T line is coated with a goat anti-feline IgG polyclonal antibody, and the control C line is coated with a mouse anti-VP2 protein monoclonal antibody, a mouse anti-VP1 protein monoclonal antibody, and a mouse anti-gD protein monoclonal antibody.

In specific examples of the present application, the SiO₂-coated quantum dot microspheres were purchased from XI'AN QIYUE BIOLOGY, Cat. No. Q0351831; the nitrocellulose membrane was of the model Unisart CN 140/1UN14E, purchased from Sartorius; the glass fiber was purchased from Shanghai Kinbio Tech Co., Ltd., with the model Ahlstrom 8964; the goat anti-feline IgG polyclonal antibody, the mouse anti-VP2 protein monoclonal antibody, the mouse anti-VP1 protein monoclonal antibody, the mouse anti-gD protein monoclonal antibody, the feline panleukopenia virus VP2 protein, the feline calicivirus VP1 protein, and the feline herpesvirus gD protein were all self-made by Beijing Jinnuo Biotech Co., Ltd.; the CAS number of thiourea is 62-56-6, and the CAS number of deferoxamine is 70-51-9; and other reagents, solvents, etc. used in the present application were commercially available.

The present application will be further described in detail below with reference to examples and performance test experiments.

### Example 1

Example 1 provided a feline triple antibody chromatographic test strip.

The method for preparing the above feline triple antibody chromatographic test strip comprised the following steps:

### (1) Preparation of Conjugate Pad

(1-1) Activation of SiO₂-coated quantum dot microspheres: 100 μmol each of SiO₂-coated quantum dot microspheres emitting at wavelengths of 610 nm, 565 nm, and 525 nm were placed in separate 1.5 ml centrifuge tubes. 150 μL of MES buffer (20 mM, pH 6.0), 40 μg of EDC, and 40 μg of NHS were sequentially added to each tube, and the mixtures were incubated in a shaker at 37° C. and 200 rpm for 20 min in the dark. The incubation mixtures were then centrifuged at 8000 g and 25° C. for 30 min, the supernatants were discarded, and the pellets were resuspended by adding 150 μL of MES buffer (20 mM, pH 6.0) to obtain resuspended solutions of activated SiO₂-coated quantum dot microspheres emitting at wavelengths of 565 nm, 525 nm, and 610 nm, respectively.
(1-2) Labeling of antigens: 20 μg of VP2 protein was added to the resuspended solution of SiO₂-coated quantum dot microspheres emitting a light at a wavelength of 565 nm; 15 μg of VP1 protein was added to the resuspended solution of SiO₂-coated quantum dot microspheres emitting a light at a wavelength of 525 nm; and 30 μg of gD protein was added to the resuspended solution of SiO₂-coated quantum dot microspheres emitting a light at a wavelength of 610 nm. The above resuspended solutions were vortexed and briefly centrifuged. The centrifuge tubes were wrapped with tin foil and placed in a shaker shaking at 200 rpm, and incubated at 37° C. for 2 hours. Then, 10 μl of 1% BSA solution was added to each centrifuge tube. The centrifuge tubes were wrapped with tin foil and placed in a shaker shaking at 200 rpm, and incubated at 37° C. for 20 min. Finally, centrifugation was performed at 8000 g and 25° C. for 15 min. The supernatants were discarded, and the pellets were resuspended with 200 μl of Tris-HCl (10 mM, pH 8.0) to obtain a solution of VP1 protein labeled with SiO₂-coated quantum dot microspheres, a solution of VP2 protein labeled with SiO₂-coated quantum dot microspheres, and a solution of gD protein labeled with SiO₂-coated quantum dot microspheres, respectively.

### (1-3) Preparation of Conjugate Pad

First, 0.5 g of sucrose, 0.3 g of trehalose, 0.05 g of BSA, and 0.03 g of gelatin were weighed and dissolved in 10 mL of Tris-HCl (100 mM, pH 8.0). After dissolution, Tween-20 and Proclin300 were added to final concentrations of 0.2% and 0.03%, respectively. Finally, the solution was filtered through a 0.22 μm membrane filter to obtain a reconstitution solution.

100 μL of the solution of VP1 protein labeled with SiO₂-coated quantum dot microspheres, 100 μL of the solution of VP2 protein labeled with SiO₂-coated quantum dot microspheres, and 100 μL of the solution of gD protein labeled with SiO₂-coated quantum dot microspheres were each diluted with 300 μL of the reconstitution solution to obtain three conjugate pad spraying solutions.

The glass fiber was marked according to a width of 1.2 cm. Under a humidity of 20%-30% and a temperature of 20° C.-25° C., a sprayer was used to first spray the spraying solution of the VP2 protein labeled with SiO₂-coated quantum dot microspheres according to the marked width, at a spraying amount of 8 μL/cm. After vacuum lyophilization at −25° C. for 24 hours, the spraying solution of the VP1 protein labeled with SiO₂-coated quantum dot microspheres was then sprayed, at a spraying amount of 8 μl/cm. After vacuum lyophilization at −25° C. for 24 hours, the spraying solution of the gD protein labeled with SiO₂-coated quantum dot microspheres was then sprayed, at a spraying amount of 8 μl/cm. After vacuum lyophilization at −25° C. for 48 hours, the conjugate pad containing VP2 protein labeled with quantum dot microspheres, VP1 protein labeled with quantum dot microspheres, and gD protein labeled with quantum dot microspheres was obtained.

### (2) Preparation of Nitrocellulose Membrane

(2-1) Preparation of a stock solution: Sucrose was dissolved in 0.01 M PBS buffer (pH 7.4) to prepare a stock solution with a final concentration of 20%, and filtered through a 0.22 μm membrane filter to obtain a stock solution.
(2-2) Preparation of diluted solution of polyclonal antibody of T line (goat anti-feline IgG polyclonal antibody): 26 μg of goat anti-feline IgG polyclonal antibody was aspirated, 5.4 μL of the stock solution was added, and the volume was made up to 36 μL with PBS buffer to obtain the diluted solution of goat anti-feline IgG polyclonal antibody.
(2-3) Preparation of diluted solutions of monoclonal antibodies of C line (mouse anti-VP1 protein monoclonal antibody, mouse anti-VP2 protein monoclonal antibody, and mouse anti-gD protein monoclonal antibody): 30 μg of mouse anti-VP1 monoclonal antibody was aspirated, 5.4 μl of the stock solution was added, and the volume was made up to 36 μl with PBS to obtain the diluted solution of mouse anti-VP1 protein monoclonal antibody. 30 μg of mouse anti-VP2 monoclonal antibody was aspirated, 5.4 μl of the stock solution was added, and the volume was made up to 36 μl with PBS to obtain the diluted solution of mouse anti-VP2 protein monoclonal antibody. 30 μg of mouse anti-gD monoclonal antibody was aspirated, 5.4 μl of the stock solution was added, and the volume was made up to 36 μl with PBS to obtain the diluted solution of mouse anti-gD protein monoclonal antibody.
(2-4) Preparation of nitrocellulose membrane: First, a test T line and a control C line were drawn on the surface of a nitrocellulose membrane (NC membrane). The test T line was 0.8 cm from one side of the NC membrane, and the control C line was 1.2 cm from one side of the NC membrane (same side), i.e., the distance between the test T line and the control C line was 4 mm.

Using a dispenser, the diluted solution of mouse anti-VP1 protein monoclonal antibody was sprayed onto the control C line, followed by vacuum drying at 37° C. for 24 h; then, the diluted solution of mouse anti-VP2 protein monoclonal antibody was sprayed onto the control C line, followed by vacuum drying at 37° C. for 24 h; and subsequently, the diluted solution of mouse anti-gD protein monoclonal antibody was sprayed onto the control C line, and the diluted solution of goat anti-feline IgG polyclonal antibody was sprayed onto the test T line, followed by vacuum drying at 37° C. for 72 h to obtain the nitrocellulose membrane. In the above spraying operation, the spraying amount was 1 μL/cm.

### (3) Preparation of Sample Pad

(3-1) Preparation of sample pad treatment solution: 3 g of bovine serum albumin and 1 g of gelatin were weighed and dissolved in 240 mL of PBS solution (0.01 M, pH 7.4). 1.25 mL of Tween-20 was added to a final concentration of 0.5%, and the volume was made up to 250 mL. The resulting solution was filtered through a 0.22 μm membrane filter to obtain the sample pad treatment solution.
(3-2) Preparation of sample pad: The glass fiber was placed in a tray. The glass fiber was wetted with the sample pad treatment solution using a pipette, soaked for 20 min, and then placed in an oven at 37° C. for drying for 24 h to obtain the sample pad.
(4) Assembly of feline triple antibody chromatographic test strip: Under a humidity of 20%-30% and a temperature of 20° C.-25° C., the prepared sample pad, conjugate pad, nitrocellulose membrane, and absorbent pad were sequentially pasted onto a PVC backing plate with an overlap of 2±1 mm. The assembly was then placed into a cutter to obtain test strips with a width of 4±0.1 mm, which were placed into cassettes to obtain the feline triple antibody chromatographic test strip.

### Comparative Example 1

Comparative Example 1 provided a feline triple antibody chromatographic test strip.

The difference between the above comparative example and Example 1 lies in that all quantum dot microspheres were not coated with SiO₂. Specifically:
the conjugate pad of Comparative Example 1 contained a feline panleukopenia virus VP2 protein probe labeled with quantum dot microspheres emitting a light at a wavelength of 565 nm, a feline calicivirus VP1 protein probe labeled with quantum dot microspheres emitting a light at a wavelength of 525 nm, and a feline herpesvirus gD protein probe labeled with quantum dot microspheres emitting a light at a wavelength of 610 nm.

### Application Example 1

Application Example 1 provided a feline triple antibody chromatographic test strip kit.

The above feline triple antibody chromatographic test strip kit comprised the feline triple antibody chromatographic test strip provided in Example 1 and a sample diluent;
the above sample diluent comprised the following components in mass content: 1% of PEG2000, 1% of Tween-20, 0.03% of Proclin300, 0.3% of thiourea, 0.1% of deferoxamine, and the balance being a 100 mM Tris-HCl buffer at pH 8.0.

### Application Examples 2-8

Application Examples 2-8 each provided a feline triple antibody chromatographic test strip kit.

The differences between the above application examples and Application Example 1 lie in: the contents of thiourea and deferoxamine in the sample diluent, as specifically shown in Table 1 below.

TABLE 1
Mass contents of thiourea and deferoxamine in sample
diluents used in Application Examples 2-8

| Application | | Deferoxamine |
|---|---|---|
| Example | Thiourea content (%) | content (%) |
| 1 | 0.3 | 0.1 |
| 2 | 0.1 | 0.1 |
| 3 | 0.2 | 0.1 |
| 4 | 0.4 | |
| 5 | 0.5 | |
| 6 | 0.3 | 0.05 |
| 7 | | 0.15 |
| 8 | | 0.2 |

### Comparative Application Example 1

Comparative Application Example 1 provided a feline triple antibody chromatographic test strip kit.

The difference between the above comparative application example and Application Example 1 lay in that the feline triple antibody chromatographic test strip used in Comparative Application Example 1 was derived from Comparative Example 1.

### Comparative Application Example 2

Comparative Application Example 2 provided a feline triple antibody chromatographic test strip kit.

The difference between the above comparative application example and Application Example 1 lay in that the sample diluent did not contain thiourea.

### Comparative Application Example 3

Comparative Application Example 3 provided a feline triple antibody chromatographic test strip kit.

The difference between the above comparative application example and Application Example 1 lay in that the sample diluent did not contain deferoxamine.

### Comparative Application Example 4

Comparative Application Example 4 provided a feline triple antibody chromatographic test strip kit.

The difference between the above comparative application example and Application Example 1 lay in that the sample diluent did not contain thiourea and deferoxamine.

### Performance Test Experiment

The feline triple antibody chromatographic test strip kits prepared in Application Examples 1-8 and Comparative Application Examples 1~4 were used to detect test samples. The detection was performed as follows. The results are shown in Table 2 below.
1. Detection method: First, 10 μL of serum/saliva was aspirated and added to 990 μL of the sample diluent, and mixed uniformly to obtain a test solution. Then, 100 μL of the test solution was aspirated and added to the sample well of the feline triple antibody chromatographic test strip, and the test strip was left to stand for 10-15 min. Finally, a fluorescence analyzer was used to read the T/(T+C) values at emission wavelengths of 565 nm, 525 nm, and 610 nm, respectively. Based on the T/(T+C) values at different emission wavelengths, whether the test sample was positive or negative was judged.

Note: The T value represents the fluorescence intensity at the test line, the C value represents the fluorescence intensity at the control line, and the T/(T+C) value is the ratio of the fluorescence intensity of the test line to the sum of the fluorescence intensities of the test line and the control line; at a given emission wavelength, when the T/(T+C) value is >0.1, it indicates that the test sample contains the antibody corresponding to the virus antigen labeled with the microspheres with that wavelength.
2. According to the above detection method, for each test sample (serum sample 1, serum sample 2, serum sample 3, and saliva sample), 30 parallel tests were performed with the kit from each example to determine whether the test sample was positive or negative. The results are shown in Table 2 below.
3. A commercial Biogal feline triple antibody rapid test kit (Dot ELISA) was used to detect serum sample 1, serum sample 2, serum sample 3, and the saliva sample. The results are shown in Table 3.

Note: It is known that serum sample 1 contains feline panleukopenia virus antibody, feline calicivirus antibody, and feline herpesvirus antibody; serum sample 2 contains feline panleukopenia virus antibody and feline calicivirus antibody; serum samples 3 contains only feline herpesvirus antibody; and the saliva sample contains feline panleukopenia virus antibody and feline herpesvirus antibody.

TABLE 2
Sample detection results of feline triple antibody chromatographic test strip kits
of Application Examples 1-8 and Comparative Application Examples 1-4

| | | Serum sample 1 | | | Serum sample 2 | | | Serum sample 3 | | | Saliva sample | | |
|---|---|---|---|---|---|---|---|---|---|---|---|---|---|
| | | 565 | 525 | 610 | 565 | 525 | 610 | 565 | 525 | 610 | 565 | 525 | 610 |
| / | | nm | nm | nm | nm | nm | nm | nm | nm | nm | nm | nm | nm |
| Application | 1 | +(30) | +(30) | +(30) | +(30) | +(30) | −(30) | −(30) | −(30) | +(30) | +(30) | −(30) | +(30) |
| Example | 2 | +(30) | +(30) | +(30) | +(30) | +(30) | −(29) | −(30) | −(29) | +(30) | +(30) | −(29) | +(30) |
| | 3 | +(30) | +(30) | +(30) | +(30) | +(30) | −(30) | −(30) | −(30) | +(30) | +(30) | −(30) | +(30) |
| | 4 | +(30) | +(30) | +(30) | +(30) | +(30) | −(30) | −(30) | −(30) | +(30) | +(30) | −(30) | +(30) |
| | 5 | +(30) | +(30) | +(30) | +(30) | +(30) | −(30) | −(29) | −(30) | +(30) | +(30) | −(29) | +(30) |
| | 6 | +(30) | +(30) | +(30) | +(30) | +(30) | −(29) | −(30) | −(29) | +(30) | +(30) | −(30) | +(30) |
| | 7 | +(30) | +(30) | +(30) | +(30) | +(30) | −(30) | −(30) | −(30) | +(30) | +(30) | −(30) | +(30) |
| | 8 | +(30) | +(30) | +(30) | +(30) | +(30) | −(30) | −(30) | −(30) | +(30) | +(30) | −(30) | +(30) |
| Comparative | 1 | +(29) | +(28) | +(27) | +(29) | +(27) | −(28) | −(30) | −(26) | +(28) | +(29) | −(29) | +(27) |
| Application | 2 | +(30) | +(30) | +(30) | +(30) | +(30) | −(29) | −(27) | −(29) | +(30) | +(30) | −(27) | +(30) |
| Example | 3 | +(30) | +(30) | +(30) | +(30) | +(30) | −(29) | −(29) | −(28) | +(30) | +(30) | −(28) | +(30) |
| | 4 | +(30) | +(30) | +(30) | +(30) | +(30) | −(27) | −(28) | −(25) | +(30) | +(30) | −(26) | +(30) |

/ Note:
+ indicates positive; − indicates negative; +(30) indicates 30 positive results, and −(30) indicates 30 negative results.

TABLE 3
Positive and negative detection results of four samples
by commercially available Dot ELISA product

| | Feline | Feline | Feline |
|---|---|---|---|
| | panleukopenia | calicivirus | herpesvirus |
| Sample type | virus antibody | antibody | antibody |
| Serum sample (1) | +(27) | +(25) | +(29) |
| Serum sample (2) | +(28) | +(26) | −(24) |
| Serum sample (3) | −(23) | −(23) | +(28) |
| Saliva sample | +(21) | −(23) | +(21) |

Based on the detection results in Table 2 and Table 3, it can be seen that the feline triple antibody chromatographic test strips provided in Application Examples 1-8 of the present application were used to detect the serum samples and the saliva sample, and the accuracy of the detection results for the three viral antibodies was 96.7%-100% (≥95%). Using commercially available Dot ELISA product for detection involved a complex operation process and relatively poor detection accuracy, especially for weakly positive samples (saliva sample), where the detection accuracy was only 70.0%-76.7% (<95%). Therefore, it is indicated that the feline triple antibody chromatographic test strips provided by the present application have the advantages of simple operation method, detection results not being subject to the subjective influence of the tester, high accuracy of detection results, good reproducibility, etc., and can be used in places such as pet hospitals, animal quarantine departments, and farms, providing strong support for disease prevention.

In Examples 1-8, by using SiO₂-coated quantum dot microspheres emitting a light at a wavelength of 565 nm to label the feline panleukopenia virus VP2 protein, SiO₂-coated quantum dot microspheres emitting a light at a wavelength of 525 nm to label the feline calicivirus VP1 protein, and SiO₂-coated quantum dot microspheres emitting a light at a wavelength of 610 nm to label the feline herpesvirus gD protein, the obtained feline triple antibody chromatographic test strips achieved a detection accuracy of 96.7%-100%.

In Comparative Example 1, by using quantum dot microspheres without SiO₂ coating to label the three proteins, the obtained feline triple antibody chromatographic test strip only achieved a detection accuracy of 86.67%-100% for the three antibodies. This indicated that the quantum dot microspheres in the above test strip exhibits fluorescence attenuation, and their fluorescence stability was poor, leading to poor accuracy of the detection results.

From the detection results of Application Examples 1-8 and Comparative Application Examples 2-4, it can be seen that in Comparative Application Examples 2-4, the samples were diluted using the sample diluent containing only one of thiourea and deferoxamine, or the sample diluent containing neither thiourea nor deferoxamine. The detection results showed that the detection accuracy for positive samples was 100%, while the detection accuracy for negative samples was 83.3%-96.7%. In Application Examples 1-8, the samples were diluted using the sample diluent containing both thiourea and deferoxamine. The detection results showed that the detection accuracy for positive samples was 100%, while the detection accuracy for negative samples was 96.7%-100%. Therefore, this indicated that diluting the samples using the sample diluent containing both thiourea and deferoxamine in the present application could reduce false positives in detection results, resulting in higher accuracy of the detection results. Further comparison revealed that in the detection results of Application Example 1, Application Examples 3-4, and Application Examples 7-8, the detection accuracy for both positive samples and negative samples was 100%, indicating that in the present application, by further controlling the content of thiourea within the range of 0.2%-0.4% and the content of deferoxamine within the range of 0.1%-0.2%, the obtained detection results have higher accuracy.

In summary, in the present application, by using SiO₂-coated quantum dot microspheres emitting a light at a wavelength of 565 nm to label feline panleukopenia virus VP2 protein, SiO₂-coated quantum dot microspheres emitting a light at a wavelength of 525 nm to label feline calicivirus VP1 protein, and SiO₂-coated quantum dot microspheres emitting a light at a wavelength of 610 nm to label feline herpesvirus gD protein, the prepared feline triple antibody chromatographic test strip can achieve effective detection of feline panleukopenia virus antibody, feline calicivirus antibody, and feline herpesvirus antibody, the detection results are not subject to the subjective influence of the tester, and the detection results have high accuracy and good reproducibility.

Although the present disclosure has been described in detail with general explanations and specific embodiments, it is obvious to a person skilled in the art that some modifications or improvements can be made thereto based on the present disclosure. Therefore, such modifications and improvements which can be made without departing from the spirit of the present disclosure belong to the scope of protection of the present disclosure.

What is claimed is:

1. A feline triple antibody chromatographic test strip kit, comprising a feline triple antibody chromatographic test strip and a sample diluent, wherein the feline triple antibody chromatographic test strip comprises a PVC backing plate; and wherein the PVC backing plate is provided with a sample pad, a conjugate pad, a nitrocellulose membrane, and an absorbent pad; and the nitrocellulose membrane is provided with a test T line and a control C line;
wherein the conjugate pad contains a feline panleukopenia virus VP2 protein probe labeled with SiO₂-coated quantum dot microspheres emitting a light at a wavelength of 565 nm, a feline calicivirus VP1 protein probe labeled with SiO₂-coated quantum dot microspheres emitting a light at a wavelength of 525 nm, and a feline herpesvirus gD protein probe labeled with SiO₂-coated quantum dot microspheres emitting a light at a wavelength of 610 nm; and
wherein the test T line is coated with a goat anti-feline IgG polyclonal antibody, and the control C line is coated with a mouse anti-VP2 protein monoclonal antibody, a mouse anti-VP1 protein monoclonal antibody, and a mouse anti-gD protein monoclonal antibody; and
the sample diluent comprises the following components in mass content: 0.8%-1.2% of PEG2000, 0.8%-1.2% of Tween-20, 0.02%-0.04% of Proclin300, 0.2%-0.4% of thiourea, 0.1%-0.2% of deferoxamine, and the balance being a 100 mM Tris-HCl buffer at pH 8.0.

2. The feline triple antibody chromatographic test strip kit according to claim 1, wherein the feline triple antibody chromatographic test strip is prepared by a process comprising the following steps:
preparing a conjugate pad;
preparing a nitrocellulose membrane;
preparing a sample pad; and
assembling the feline triple antibody chromatographic test strip;
wherein the conjugate pad is prepared by the following process:
activating SiO₂-coated quantum dot microspheres emitting at wavelengths of 565 nm, 525 nm, and 610 nm;
respectively adding feline panleukopenia virus VP2 protein, feline calicivirus VP1 protein, and feline herpesvirus gD protein for antigen labeling; and
performing blocking and resuspension to respectively obtain a solution of VP2 protein labeled with SiO₂-coated quantum dot microspheres, a solution of VP1 protein labeled with SiO₂-coated quantum dot microspheres, and a solution of gD protein labeled with SiO₂-coated quantum dot microspheres;
diluting the solution of VP2 protein labeled with SiO₂-coated quantum dot microspheres, the solution of VP1 protein labeled with SiO₂-coated quantum dot microspheres, and the solution of gD protein labeled with SiO₂-coated quantum dot microspheres to a final concentration of 10 v/v % to 20 v/v %; and
sequentially spraying the diluted solutions onto a glass fiber surface at a spraying amount of 8±1 μL/cm to obtain the conjugate pad containing VP2 protein labeled with SiO₂-coated quantum dot microspheres, VP1 protein labeled with SiO₂-coated quantum dot microspheres, and gD protein labeled with SiO₂-coated quantum dot microspheres.

3. The feline triple antibody chromatographic test strip kit according to claim 2, wherein the nitrocellulose membrane is prepared by the following process:
first, drawing a test T line and a control C line on the surface of a nitrocellulose membrane; and then, using a dispenser;
sequentially spraying the diluted solution of mouse anti-VP2 protein monoclonal antibody, the diluted solution of mouse anti-VP1 protein monoclonal antibody, and the diluted solution of mouse anti-gD protein monoclonal antibody onto the control C line; and
spraying the diluted solution of goat anti-feline IgG polyclonal antibody onto the test T line, followed by vacuum drying to obtain the nitrocellulose membrane;
wherein the concentrations of the diluted solution of mouse anti-VP2 protein monoclonal antibody, the diluted solution of mouse anti-VP1 protein monoclonal antibody, and the diluted solution of mouse anti-gD protein monoclonal antibody are each 0.95-1.05 μg/μL; the concentration of the diluted solution of goat anti-feline IgG polyclonal antibody is 0.75-0.85 μg/μL; the spraying amounts are each 0.8-1.2 μL/cm.

4. The feline triple antibody chromatographic test strip kit according to claim 3, wherein in the nitrocellulose membrane, a distance between the test T line and the control C line is set at 4±0.5 mm.

5. The feline triple antibody chromatographic test strip kit according to claim 3, wherein in the spraying operation of the control C line, after one diluted solution is sprayed, vacuum drying is performed, and then another diluted solution is sprayed.

6. The feline triple antibody chromatographic test strip kit according to claim 5, wherein in the spraying operation of the control C line, a temperature of the vacuum drying is set at 37° C.±5° C., and a time of the vacuum drying is set at 24-72 h.

7. The feline triple antibody chromatographic test strip kit according to claim 2, wherein the sample pad is prepared by the following process: first, preparing a sample pad treatment solution, then soaking a glass fiber in the sample pad treatment solution for 20-25 min, and drying the glass fiber to obtain the sample pad; and
wherein the sample pad treatment solution is prepared by the following process: dissolving bovine serum albumin and gelatin in a PBS solution, then adding Tween-20, and filtering the resulting solution to obtain the sample pad treatment solution.
